(19) United States
(12) Patent Application Publication
(10) Pub. No.: US 2009/0041674 A1
(43) Pub. Date: Feb. 12, 2009

(54) TARGETED IRON OXIDE NANPARTICLES

(76) Inventors: William Alexander Jones, London (GB); Kishore Kumar Bhakoo, London (GB)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/280,321

(22) PCT Filed: Feb. 14, 2007
(86) PCT No.: PCT/GB2007/000526

§ 371 Date: Aug. 21, 2008

Related U.S. Application Data
(60) Provisional application No. 60/743,363, filed on Feb. 28, 2006.

(30) Foreign Application Priority Data
Feb. 28, 2006 (GB) 0604009.1

(51) Int. Cl. A61K 49/18; C07F 15/02; G01N 33/543

(52) U.S. Cl. 424/9.322; 556/138; 556/32; 536/18.7; 530/322; 424/9.32; 435/29

(57) ABSTRACT

Iron oxide nanoparticles comprising functional groups of the formula —X—NH₂ on the outer surface of said nanoparticles, wherein X is selected from O, NR, NH or S, where R is C₁₋₇ alkyl and conjugate particles comprising an iron oxide nanoparticle linked via a bond of formula —C═N—X— to a cell targeting ligand, where X is selected from O, NR, NH or S, where R is C₁₋₇ alkyl.

The present invention relates to iron oxide nanoparticles suitable for conjugation to targeting ligands, and to the use of such conjugated nanoparticles in cell labelling.

### BACKGROUND

Many types of carbohydrate-containing cell-targeting molecules (ligands) are known. Examples include glycoproteins, glycolipids, polysaccharides, oligosaccharides and viral proteins. These ligands possess carbohydrate moieties which may assist in the targeting functionality, or may be merely part of the construct.

A particularly important class of carbohydrate-containing ligands are glycoproteins. These are macromolecules composed of a protein and a carbohydrate (an oligosaccharide). The carbohydrate chains can be attached at either asparagine (termed N-glycosylation) or at hydroxylysine, hydroxyproline, serine, or threonine (termed O-glycosylation). Possible carbohydrates which may be included in these chains include glucose, glucosamine, galactose, galactosamine, mannose, fucose, and sialic acid.

Glycoproteins are important for immune cell recognition, especially in mammals. For example, antibodies are glycoproteins produced by the immune system which recognise specific foreign substances (antigens) and bind to them. The antigens may be on the surface of a cell, in which case binding can lead to cell aggregation and destruction.

The antibody-antigen interaction has been used for many years as a means of targeting and labelling specific cells in complex multicellular systems and to study the cell-surface distribution of particular antigens (e.g. Raff et al., Immunology, 1970, 637; Taylor et al., Nature New Biol. 1971, 225). A critical requirement is that the appropriate labels (e.g. fluorescent dyes, radioactive markers, magnetic particles) can be attached to the antibodies in such a way as to maintain the binding activity of the antibodies.

Immunological labelling techniques employing monoclonal antibodies have been used to identify and localise specific cells in neural tissues (e.g. Eisenbarth et al., Proc. Natl. Acad. Sci., 1979, 4913). Immunological methods have also been applied to the separation of different types of cells—cells labelled with fluorescent antibodies have been separated from unlabelled cells by fluorescence-activated cell sorting (e.g. Steinkamp et al., Rev. Sci. Instrum., 1973, 1301).

More recently, the use of magnetic nanoparticles for cell targeting has been of interest. Antibodies or other targeting molecules, such as co-factors, hormones and proteins, can be attached to nanoparticles allowing targeting to specific cell types.

Nanoparticles can be utilised in Magnetic Resonance Imaging (MRI) diagnostics, as contrast agents to enhance the scanning resolution. Agents used for this purpose are most usually paramagnetic gadolinium chelates (Gadodiamide—OMNISCAN, Winthrop Pharm.; Gadoteridol—PROHANCE, Squibb) or superparamagnetic iron oxide nanoparticles (Ferumoxtran-10—COMBIDEX, Advanced Magnetics). Iron oxide nanoparticles have the advantage of lower potential for toxicity in vivo. Clinical use of these particles as MRI contrast agents in the past has generally relied on ‘passive targeting’, where the nanoparticles accumulate due to a difference in tissue permeability. More recently, advances toward ‘active targeting’ using labelling techniques, such as those discussed above, have been made.

For example, monocrystalline iron oxide nanoparticles (MION) have been conjugated to the protein transferrin for targeting of tumour cells for MRI (Högemann et al., Bioconjugate Chem., 2000, 941-946) and pancreatic receptor function has been monitored by MRI using MION particles indirectly conjugated to the peptide secretin (Shen et al., Bioconjugate Chem., 1996, 311-316). Examples of detecting specific cancers, involving decorating the surface of the nanoparticles with antibodies, have included rectal carcinoma (Toma et al., Br. J. Cancer, 2005, 93(1) 131-136), carcinoembryonic antigen (Tiefenauer et al., Magn. Reson. Imaging, 1996, 14(4), 391-402) and small cell lung carcinoma (Go et al., Eur. J. Radiol., 1993, 16(3), 171-175).

Cell labelling with magnetic nanoparticles is also of use for cell separation purposes. For example, immunospecific iron oxide particles have been prepared by conjugation to protein A from Staphylococcus aureus, then used to visualise cell surface antigens by electron microscopy and to magnetically separate labelled cells (Molday et al., J. Immunol. Methods, 1982, 353-367).

Such magnetic nanoparticles as discussed above are typically produced with a stabilising layer, which is often a polysaccharide such as dextran. This layer prevents agglomeration and improves the bio-compatibility of the particles. It can also provide sites for functionalisation of the nanoparticle surface, enabling antibodies or other molecules to be attached.

Conjugation of a glycoprotein to a labelling moiety, such as a coated nanoparticle, can be effected in a number of ways. The amino groups of lysine residues in the protein can be used to attach the glycoprotein to the labels. However this has many disadvantages. In particular, in the case of antibodies, the lysine residues are typically distributed homogenously throughout the antibody, resulting in the antigen-targeting region (variable region) becoming blocked and causing a decrease in binding efficiency. Also, many steps are often needed to perform the conjugation.

It is therefore thought to be preferable to perform conjugation via the constant region of the antibody as this is not involved in binding. Most antibodies are glycosylated in this region, and the carbohydrate residues can therefore be selectively oxidised, producing aldehyde groups. Site-directed conjugation in the constant region can then be effected via imine formation with amino groups on the labelling moiety, as shown in Scheme 1.

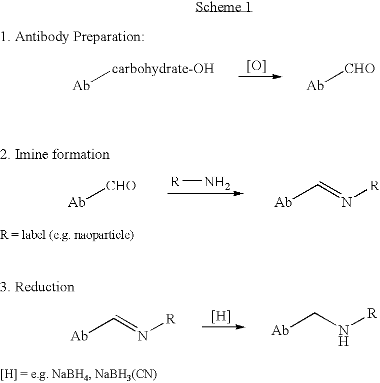

The initially formed imine bond is unstable in aqueous conditions and so it is typically reduced to a secondary amine in situ. The disadvantage of this method is that the reducing agent used (e.g. sodium borohydride) can also reduce the starting aldehyde. This can be avoided to an extent by use of the milder reducing agent sodium cyanoborohydride, but this may generate toxic cyanide by-products. Another problem with the technique is that it is not selective: amines from lysine residues on the same or different antibodies, as well as those from the labelling moiety can react with the aldehydes.

Fuentes et al. have recently investigated different conjugation procedures for immobilising antibodies on various supports, including on magnetic nanoparticles (Fuentes et al., Biosensors & Bioelectronics, 2005, 1380-1387). They showed that antibodies conjugated via the glycosylated region to amine-functionalised nanoparticles retained nearly all of their initial recognition capacity. The conjugate particles needed to be kept in physiological salt solutions to maintain high affinity over time.

### SUMMARY OF THE INVENTION

The present inventors have now produced iron oxide nanoparticles which show improved stability in physiological salt solutions and have developed an improved method for the conjugation of carbohydrate-containing ligands, preferably glycoproteins such as antibodies, to nanoparticles.

A first aspect of the present invention provides iron oxide nanoparticles comprising —X—NH₂ functional groups on their outer surface, where X is a divalent heteroatom residue such as O, S NH, or NR, where R is C₁₋₇ alkyl. These functional groups can act as reactive sites for attachment of cell-targeting ligands to the nanoparticles.

The nanoparticles of the first aspect may optionally comprise a coating of cross-linked polysaccharide. Such a coating is thought to improve the physiological stability of the iron oxide nanoparticles.

A second aspect of the present invention provides a method for the synthesis of the iron oxide particles of the first aspect. In particular, the synthesis may proceed via intermediate iron oxide nanoparticles comprising functional groups of formula —X—NR¹R² on their outer surface, wherein R¹ is H and R² is an amine protecting group, or where R¹ and R² may together form an amine protecting group.

A third aspect of the present invention provides a process of attaching carbohydrate-containing cell-targeting ligands to nanoparticles of the first aspect of the invention, by reaction of aldehyde groups on said ligands with said functional groups on the nanoparticle surface. The aldehyde groups may be produced by selective oxidation of the carbohydrate-containing ligand. This reaction produces a double bond (—C═N—X—) between the antibody and the nanoparticle. This bond is less reactive to nucleophilic attack than the corresponding double bond in an imine, as produced in the previous conjugation method, which results in greater stability in aqueous conditions. This aspect of the present invention removes the need for a subsequent reduction step.

A fourth aspect of the present invention provides an ligand-nanoparticle conjugate, comprising a carbohydrate-containing cell-targeting ligand, which is preferably a glycoprotein, and most preferably an antibody, bound to an iron oxide nanoparticle by a double bond as described above. These conjugates are preferably produced by the method of the third aspect.

A fifth aspect of the present invention provides the use of the ligand-nanoparticle conjugates of the fourth aspect, for targeting and labelling cells. The use of these conjugates may or may not involve introducing them into the human or animal body. In the case where they are not introduced into the body, the use may be termed in vitro, i.e. reproduction of a biological process in a more easily defined environment such as a reaction vessel, culture vessel, or plate.

### DEFINITIONS

Iron oxide nanoparticles: In the context of this application, the phrase ‘iron oxide nanoparticle’ refers to sub-microscopic particles whose overall hydrodynamic diameter is less than 1 μm, as measured by dynamic light scattering (DLS), comprising a core selected from one or more of iron hydroxide, iron oxides, iron oxide hydrates, iron mixed oxides or iron, where the core is surrounded by a stabilising coating.

Stabilising coating: The stabilising coating is a layer which forms the outer surface of the nanoparticle, and which prevents agglomeration of the particles and serves to improve the biocompatibility of the particles. The coating is preferably composed of a polysaccharide.

The phrase ‘Ficoll-stabilised iron oxide nanoparticle’ refers to iron oxide nanoparticles as above, wherein the stabilising coating is composed of the highly branched co-polymer of sucrose and epichlorohydrin, sold as FICOLL.

The Ficoll coating on the nanoparticles may be cross-linked, for example by reaction with epichlorohydrin and sodium hydroxide, and the resultant nanoparticles may be referred to as cross-linked Ficoll stabilised iron oxide nanoparticles. The cross-linked Ficoll-stabilised iron oxide nanoparticles may be further reacted, with epichlorohydrin and ammonia, to provide amino (—NH₂) groups on the surface of the particles. The nanoparticles thus produced are referred to as aminated cross-linked Ficoll-stabilised iron oxide nanoparticles.

The phrase ‘functionalised nanoparticle’, in this context, refers to iron oxide nanoparticles which have been modified to provide —X—NR¹R² functional groups on the surface of the particles, wherein R¹ and R² may independently be H or amine protecting groups, or where R¹ and R² may together form an amine protecting group. Examples of functionalised nanoparticles include aminooxy functionalised nanoparticles (X═O), hydrazine functionalised nanoparticles (X═NH, NR¹, where R is C₁₋₇ alkyl) and aminosulfide functionalised nanoparticles (X═S). Preferably, the nanoparticles are aminooxy functionalised and contain the —O—NR¹R² group, which may also be described as a hydroxylamine functional group.

Amine protecting groups: Protecting groups (also known as masking groups or blocking groups) are groups which can be attached to a reactive functional group, such as an amine, in order to prevent reaction of that group. By protecting a reactive functional group, reactions involving other unprotected reactive functional groups can be performed, without affecting the protected group; the protecting group may be removed, usually in a subsequent step, without substantially affecting the remainder of the molecule. See, for example, Chapter 7 of Protective Grours in Organic Synthesis (T. Green and P. Wuts, Wiley, 1999), which is incorporated herein by reference. Preferably, the amine protecting group is one which is suitable for removal without affecting nanoparticle stability, such as a substituted imide, for example dimethylimide (—N═CMe₂) or N succinimide; or an amide, for example ^{t}butoxy amide (N—CO—OC(CH₃)₃, NBoc). In some embodiments, it is preferred that the amine protecting group is one which is suitable for removal in the presence of a glycoprotein and/or an antibody.

C₁₋₇ alkyl: The term C₁₋₇ alkyl as used herein, means a monovalent moiety obtained by removing a hydrogen atom from a C₁₋₇ hydrocarbon having from 1 to 7 carbon atoms, which may be aliphatic or alicyclic, or a combination thereof, and which may be staurated, partially unsaturated or fully unsaturated. Examples of C₁₋₇ alkyl groups include, but are not limited to, methyl, ethyl, n-propyl, iso-propyl.

Ligand: In the context of this application, the term ligand refers to a cell-targeting molecule, that is a molecule which can bind to receptors on the surface of cells. Preferably, the molecules are carbohydrate-containing cell targeting molecules, and may be glycoproteins, glycolipids, polysaccharides, oligosaccharides (for example sialyl Lewis X) and viral proteins. More preferably, they are glycoproteins and are most preferably antibodies.

Glycoprotein: a glycoprotein is a macromolecule composed of a protein and a carbohydrate (an oligosaccharide). The carbohydrate chains can be attached at either asparagine (termed N-glycosylation) or at hydroxylysine, hydroxyproline, serine, or threonine (termed O-glycosylation). Possible carbohydrates in the carbohydrate chains include glucose, glucosamine, galactose, galactosamine, mannose, fucose, and sialic acid. Glycoproteins are also called glycosylated proteins.

Antibody: An antibody is a protein produced by the human or animal body's immune system which binds to a specific antigen. Antibodies can be defined as having a ‘variable region’, which is the region where binding to the specific antigen occurs, and a ‘constant region’ which is not involved in binding. Most antibodies are glycoproteins, and are glycosylated in the constant region, which means that sugar chains are attached to the amino acid residues of the protein in this region, and it is these antibodies which are suitable for use in the present invention. The term antibody also includes antibody fragments, where the variable region is present along with a region suitable for binding to the nanoparticle.

Conjugation: Conjugation is the process of linking a ligand to a label such as a nanoparticle by formation of a chemical bond between them. The particles produced, with the ligand attached, are referred to herein as conjugate particles or conjugates.

Selectively oxidise: In the context of this application, the term selectively oxidise refers to the process of oxidising a carbohydrate-containing ligand under sufficiently mild conditions that hydroxy groups on the carbohydrate residues are oxidised to the corresponding aldehydes, but not fully oxidised (i.e. to carboxylic acids). Other functional groups which may be present in the molecule are preferably not oxidised under these conditions.

Activating group: This refers to a functional group which renders a carbonyl group to which it is attached more reactive to nucleophilic attack than the corresponding carboxylic acid (—C(O)OH). A carbonyl group may be activated as an acid chloride (—C(O)Cl), ester (—C(O)OR), 1-hydroxybenzotriazole (HOBt) derivative, mixed anhydride (—C(O)OC(O)R, e.g. —OC(O)OCH₂CH(CH₃)CH₃) or symmetrical anhydride, or N-hydroxysuccinimide derivative. Preferably the activated carbonyl compound has a relatively long half-life in aqueous solutions, and is most preferably an N-hydroxysuccinimide ester.

Physiological conditions: The phrase ‘physiological conditions’ refers to conditions such as those found inside a cell or whole body circulation. These conditions can be defined as being aqueous, with pH close to physiological pH (7.4) and with the presence of dissolved salts, for example sodium, potassium, calcium and magnesium salts.

### DETAILED DESCRIPTION OF THE INVENTION

### Functionalised Nanoparticles

As discussed above and as known to those skilled in the art, the most desirable way to attach a moiety, such as a magnetic nanoparticle, to an antibody is via the constant region. This can be done by selective oxidation of the glycosyl residues on the antibody, using a mild oxidant such as sodium periodate, to produce aldehyde residues which can then be reacted with nucleophilic functional groups on the labelling moiety (Fuentes et al, Biosensors & Bioelectronics, 2005, 20, 1380-1387; Molday et al, J. Immunol. Meth. 1982, 52, 353-367; Sanderson et al, Immunology, 1971, 20, 1061-1065). This has been shown to retain full functionality of the antibody (Abraham et al, J. Immunol. Meth., 1991, 144, 77-86). This technique is also suitable for attachment of labelling moieties to other glycosylated proteins and to other carbohydrate-containing ligands.

In answer to the need for alternative conjugation methods, functionalised iron oxide nanoparticles have been synthesised. The —X—NH₂ functional group is known to react with aldehydes to form the double bond —C═N—X—. Unlike the imine bonds formed by the analagous reaction with amine groups, the bond formed in this case is stable in physiological conditions, thus avoiding the need for the use of a reducing agent.

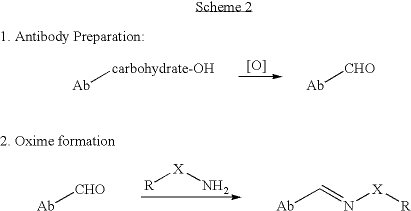

In addition, some carbohydrate-containing ligands, such as oligosaccharides, for example sialyl Lewis X, may be directly attached via the reducing end of the carbohydrate chain.

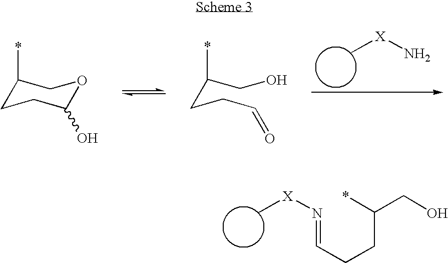

The —X—NH₂ functionality can be introduced onto the surface of iron oxide nanoparticles by amination of the nanoparticles' stabilising coating, with epichlorohydrin and ammonia, and then reaction with a linker such as compound IV (Scheme 3).

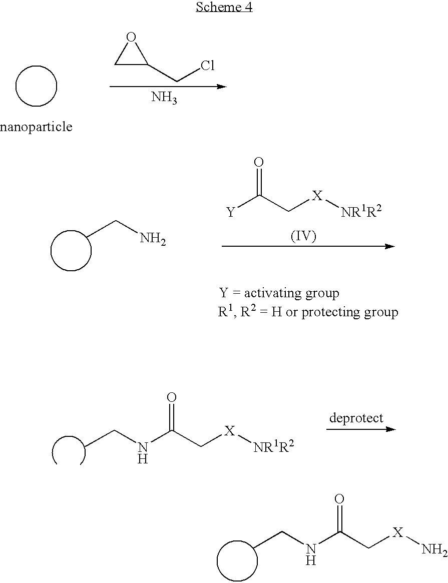

The linker IV is an activated ester containing a protected hydroxylamine, hydrazine or aminosulfide group, for example the N-hydroxysuccinimide ester IVa where Y is a N-hydroxysuccinimide group and R¹ and R² together form a dimethylimide protecting group.

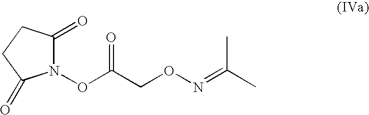

The linker of formula IV (and IVa) is illustrative of a class of linkers suitable for use in the present invention, which are of formula 4:

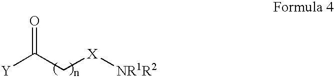

where:
Y is an activating group;
R¹ and R² are independently selected from H and an amine protecting group, or together may form an amine protecting group;
X is as defined in the first aspect of the invention (i.e. O, S, NH or NR) and
n is 1 to 7.

Such linkers result in the functionalised nanoparticles being of formula 5:

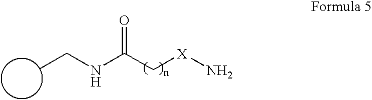

and the conjugated particles being of formula 6:

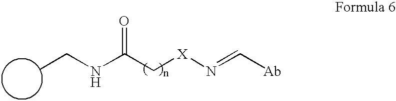

The functionalised nanoparticles provided by the above method are suitable for direct reaction with aldehyde-containing ligands, for example with prepared (oxidised) glycoproteins. This represents a considerable advantage over presently commercially available nanoparticles, which require prior chemical modification, often involving several steps, before the glycoprotein can be attached.

Once a ligand such as an antibody is attached, the ligand-nanoparticle conjugates can be administered to patients and used for in vivo cell tracking—the magnetic nanoparticles are incorporated into cells targeted by the ligands in the conjugates and these can be localised by MRI. For example, in areas where cells are targeted with ligand-labelled iron oxide nanoparticles, there is a local change in signal intensity, attributed to a local change in T2 (transverse) relaxation time. The overall effect is a hypointense region where the iron oxide has accumulated. Cells of interest are therefore located in areas of hypointense regions.

Another use is in the separation of cells—the ligand-nanoparticle conjugates can be used to target specific cell types and separation of these cells can then be effected using a magnetic field. Ligand-nanoparticle conjugates bind specifically to cells presenting antigens which are recognised by the ligand. Separation of the labelled cells is then effected by passing the cells through a magnetisable column (e.g. Macs column, Miltenyi Biotech). Labelled cells are retained by the magnetic field whilst non-labelled cells pass through the column.

Another promising medical application is the use of the nanoparticles as a means to deliver a drug to a particular site using magnetic guidance. The nanoparticles are conjugated to a drug molecule, which contains a selectively oxidised carbohydrate group or other aldehyde, and administered to a patient. The drug is concentrated at the desired site on local application of a magnet. This particular application would be suitable for the local delivery of cytotoxic agents, as is the case in cancer treatment, and is adaptable to a variety of agents which are susceptible to incorporation of a carbohydrate or aldehyde group. This technology is being investigated with other platforms (Eur Biophys J., 2006, Jan 31; 1-5)

Other medical applications related to those described above include use as a haematinic agent to treat anaemia and thermal treatment of cancers by magnetic fluid hyperthermia (Prostate, 2006, Jan 1; 66(1): 97-104).

### Production of Nanoparticles

The nanoparticles of the present invention can be produced in a variety of sizes (20-120 nm) as measured by dynamic light scattering (DLS). The size can be controlled and the optimum size can be tailored to a specific purpose. Generally, where increased magnetic moment is required, larger particles are preferable.

The methodology for the conjugation process was tested by reaction of a selectively oxidised antibody with a fluorescent marker containing a hydrazine functional group, as well as by the conjugation of a fluorescently labelled antibody to a nanoparticle.

### Stabilising Coating

As discussed above, Fuentes et al found that antibody-nanoparticle conjugates needed to be stored in physiological salt solutions to maintain antibody affinity. However, DVLO theory (as independently developed by Derjaguin and Landau (Acta Physicochim, URSS, 1941 14, 633), and Verwey and Overbeek (Theory and Stability of Lyophobic Colloids: The Interaction of Sol Particles Having an Electrical Double Layer. Elsevier, Inc, 1948)) predicts that colloidal stability is decreased in salt solutions.

The Ficoll-stabilised iron-oxide nanoparticles useful in the present invention, however, have been found by the present inventors to be stable in such solutions and are therefore suitable for long term storage.

To overcome inherent nanoparticle instability over an extended period of time in physiological conditions, a coating procedure was employed using a highly branched co-polymer of sucrose and epichlorohydrin. Without wishing to be bound by theory, it was rationalised that the highly branched nature might increase the nanoparticle stability

The iron oxide nanoparticles can be produced from a mixture of iron (II) chloride and iron (III) chloride with commercial polysaccharide (FicoII™) in water, by treatment with base (e.g. NaOH or NH₄OH) and heating under an inert atmosphere. Subsequent cross-linking, followed by further reaction with epichlorohydrin produces reactive sites on the surface which can then be aminated by treatment with ammonia or an amine such as 1,2-bis(2-aminoethoxy)ethane.

The concentration of amine groups on the surface can be determined by treating the nanoparticles with fluorescamine, which reacts with primary amine groups, producing derivatives whose fluorescence is proportional to the number of amine groups.

The stability of the nanoparticles in physiological solution can also be determined. The particles of the present invention exemplified below are found to be stable in physiological buffer and were unaltered after 41 days.

### BRIEF DESCRIPTION OF THE FIGURES

FIG. 1 is a standard fluorescence curve produced by serial dilution of a 0.1 M solution of taurine.

FIG. 2 is a size exclusion chromatography (SEC) chromatogram for the nanoparticles produced in Example 1 (Toyopearl 65F column, eluant=0.15M NaCl, 0.01M NaCO₃, pH 9, flow rate 0.75 ml/min).

FIG. 3 shows the size distribution, at two time points, of Ficoll coated nanoparticles.

FIG. 4 is a size exclusion chromatography (SEC) chromatogram for the nanoparticles produced in Example 2(a)(i) (Toyopearl 65F column, eluant=0.15M NaCl, 0.01 M NaCO₃, pH 9, flow rate 0.75 ml/min).

FIG. 5 is a pH titration curve for the ammonia addition in Example 2(a).

FIG. 6 is a size exclusion chromatography (SEC) chromatograms for the nanoparticles produced in Example 2(a)(ii) (Toyopearl 65F column, eluant=0.15M NaCl, 0.01 M NaCO₃, pH 9, flow rate 0.75 ml/min)

FIG. 7 is size exclusion chromatography (SEC) chromatogram for the nanoparticles produced in Example 2(b) (Toyopearl 65F column, eluant=0.15M NaCl, 0.01M NaCO₃, pH 9, flow rate 0.75 ml/min).

FIG. 8 is size exclusion chromatography (SEC) chromatogram for the fluorescent labelled antibodies of Example 2(g) (phenomenex s2000 column, eluant=0.15M NaCl, 0.01 M NaCO₃, pH 9, flow rate 1 ml/min; fluorescent detection Ex=490 nm/Em=520 nm).

FIGS. 9 and 10 shows the GC-MS over time of the deprotection product of isopropylidene protected aminooxy functionalised nanoparticles.

FIG. 11 shows the mass-selective detection of the deprotection product of isopropylidene protected aminooxy functionalised nanoparticles.

FIG. 12 is a GC-MS of the synthesis products of 6-isopropylideneaminooxy-hexanoic acid when produced using a Leibig condenser.

FIG. 13 is a GC-MS of the synthesis products of 6-isopropylideneaminooxy-hexanoic acid when produced using microwave heating.

FIG. 14 is a fluorescent trace of an antibody-oxide reaction mixture analysed by SEC on Superose™6

### EXAMPLES

### General Methods

All reagents and solvents were the highest commercially available grades and were used without further purification. Iron (III) chloride hexahydrate (FeCl_{3.6}H₂O), iron (II) chloride tetrahydrate (FeCl₂.4H₂O), (aminooxy)acetic acid hemihydrochloride, N,N′-dicyclohexylcarbodiimide (DCC), N-hydroxysuccinimide (NHS), epichlorohydrin, sodium periodate, Toyopearl 65F, MOPC 21, triethylamine, diisopropylethylamine, hydrochloric acid and acetone were purchased from Sigma. N²-hydroxyethylpiperazine-N-2′-ethanesulfonate (HEPES), Fluorescamine, FicoII™ 400 and 1,2-bis(2-aminoethoxy)ethane were purchased from Fluka. Sodium hydroxide and concentrated ammonia solution were purchased from BDH. Deoxygenated water was produced by multiple freeze-thaw cycles under high vacuum.

Dextran-coated iron oxide nanoparticles were prepared by literature methods (Molday et al; J. Immunol. Methods; 1982; 353-367).

### Analytical Methods

### Determination of Concentration of Amine Groups/G of Nanoparticles

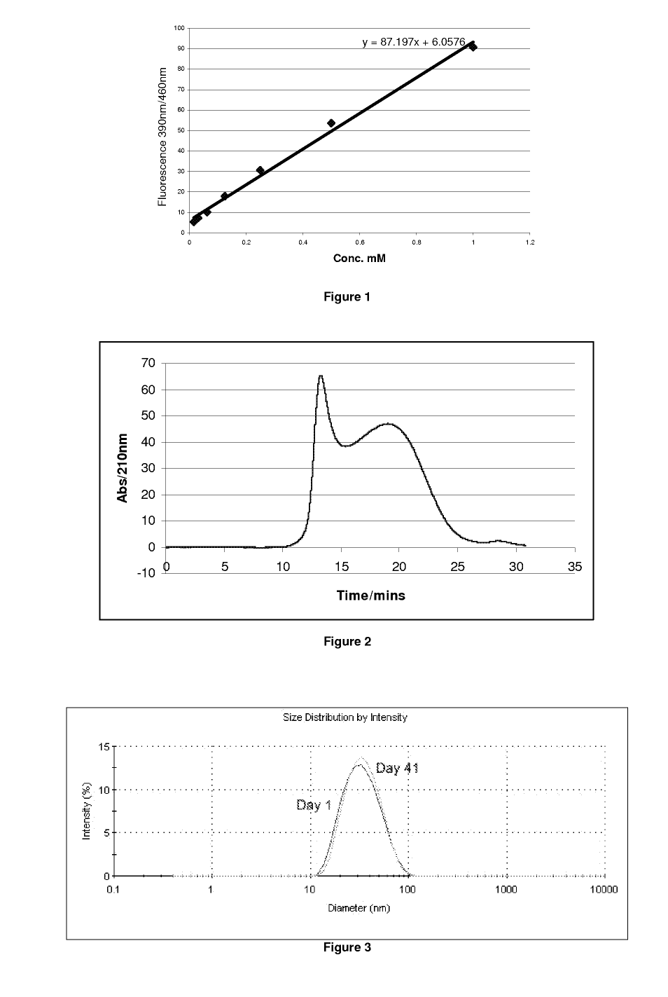

To determine the concentration of primary amine groups available for subsequent modification, the fluorescamine assay was developed. A solution of amine-functionalised iron oxide (97.5 μL) was aliquoted into 96-well plates, to which borate buffer (0.1 M Sodium borate, pH 8.5, 7.5 μL) was added. A 0.1% (w/v) solution of fluorescamine (45 μL) was added to the buffered salt solution and allowed to react for 5 minutes. A standard curve was produced by serial dilutions of a 0.1 M solution of taurine (FIG. 1). The fluorescence of the samples was measured and converted to an amino group concentration by comparison with the standard curve. Samples were freeze dried and weighed and a concentration (mmol) of primary amine per gram was calculated. Fluorescence was read on a 96-well fluorimeter, ex=390 nm, em=460 nm.

### Determination of Nanoparticle Stability

Nanoparticles (II and III) were dialysed extensively for 2 days. Verification that the salts had been dialysed out was confirmed by monitoring the solution conductivity. A 10×HEPES buffered salt solution (1.5M NaCl, 0.1 M HEPES, pH 7.2) was prepared and diluted 1:9 with the iron oxide nanoparticle solutions (II and II) and with a preparation of dextran coated iron oxide. Particle size was measured by DLS at various time points. If the particles are stable, no change in average particle size is observed. Unstable particles agglomerate and precipitate out of solution over time.

### GC-MS Method (for Example 3(d))

A sample of the reaction mixture is aliquoted into a reaction vial using acetone as the solvent. The sample is then injected into a GC-MS (1 μL, dilution 1:20)(Agilent Technologies). During injection, the column temperature is kept at 70° for 3 minutes and then ramped up to 200° C. over 20 minutes.

### Example 1

### One Pot Synthesis of Aminated, Cross-Linked Ficoll-Stabilised Iron Oxide Nanoparticles (IIa)

FeCl₃.6H₂O (0.32 g, 1.2 mmol), FeCl₂.4H₂O (0.12 g, 0.6 mmol) and FicoII™ 400 (1 g, 0.0025 mmol) were dissolved in deoxygenated water (10 ml). The iron-Ficoll solution was quickly syringed into a 1 M solution of NaOH (20 ml) heated to 50° C. under N₂. On addition, the solution immediately turned black indicating formation of iron oxide. The solution was stirred for 1 hour under N₂. After 1 hour, heat was removed, and NaOH (2.8 g) was dissolved in the iron oxide solution, followed by addition of epichlorohydrin (3.0 ml). The suspension produced was stirred overnight.

To the resulting solution was added NaOH (1.4 g, 35 mmol) and epichlorohydrin (3.0 ml, 27 mmol). After reacting for 30 minutes, concentrated ammonia solution (15 mL) was added and reaction proceeded overnight. The solution was then neutralised with 20% hydrochloric acid and concentrated by rotary evaporation to 5 ml. Size exclusion chromatography (SEC) was then effected on a 10 mm×300 mm column packed with Toyopearl 65F eluted with 0.15M NaCl, 0.01 M NaCarbonate, pH9 at 0.75 ml/min. 1.5 ml fractions were collected and fractions coloured dark brown were kept for particle sizing by Dynamic Light Scattering (DLS) using a Malvern Nano ZS.

Subjecting the samples to size exclusion chromatography (FIG. 2) allowed particles to be sorted into narrow size ranges and collected. Table 1 indicates the relationship between particle size and elution time. The polydispersity index is an indication of the size distribution within a given fraction.

TABLE 1

| Time/mins | Z(Ave) Diameter/nm | Polydispersity Index |
|---|---|---|
| 12 | 106 | 0.166 |
| 14 | 73 | 0.130 |
| 16 | 50 | 0.158 |
| 18 | 47 | 0.132 |
| 20 | 44 | 0.210 |

### Particle Stability

It was found that Ficoll-stabilised iron oxide nanoparticles were stable in isotonic buffer (0.15M NaCl, 0.01 M HEPES, pH 7.2). (FIG. 3). The hydrodynamic diameter of Ficoll-stabilised iron oxide nanoparticles remained unaltered beyond the 41 days of testing (FIG. 3).

### Example 2

### a) Synthesis of Ficoll-Stabilised Iron Oxide Nanoparticles (I)

### (i) Formation of Nanoparticles

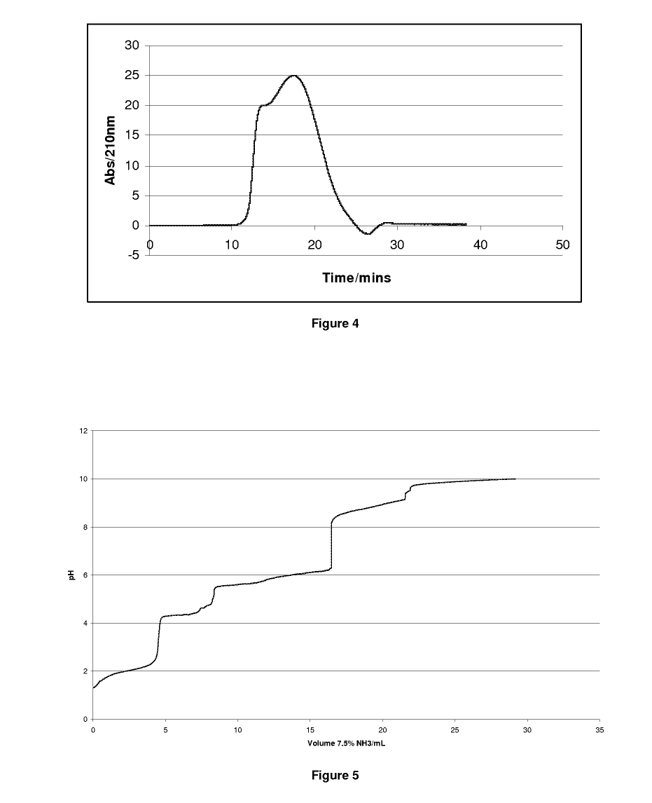

To a 15 mL solution of FeCl₃.6H₂O (1.13 g, 4.2 mmol), FeCl₂.4H₂O (0.48 g, 2.4 mmol) and FiCoII™ 70 (3.75 g, 0.053 mmol) was added ammonia (7.5% solution) dropwise to pH 10. Titration was carried out on an autotitrator, delivering 10 μl of 7.5% ammonia solution every 2 seconds. Several pH endpoints were observed during the titration of 7.5% ammonia (FIG. 5), possibly suggesting several forms of iron oxide being formed during the process. The solution was then heated at 50° C. for 1 hour. The resulting black solution was then centrifuged for 15 minutes at 1500 rpm. The supernatant was kept and the precipitate was discarded. The solution was finally filtered through 0.45 μm filters. To remove unbound FicoII™ 70, the solution was subjected to size exclusion chromatography (SEC) in 300 μl portions, on a column (10 mm×300 mm) packed with Toyopearl 65F. The resulting black/brown fractions were used for size determination by dynamic light scattering (DLS). A range of particle sizes was produced as shown in FIG. 4.

### (ii) Cross-Linking of Ficoll™ Coating

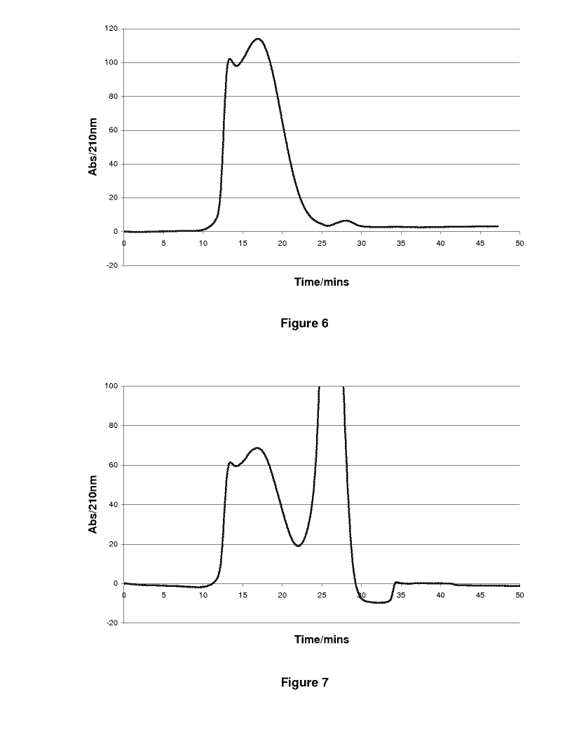

To a solution of pure Ficoll-stabilised iron oxide nanoparticles (I, 30 ml 0.3 mmol) was added sodium hydroxide (3.6 g, 90 mmol). Once the sodium hydroxide had dissolved, epichlorohydrin (3 ml, 27 mmol) was added and the resulting suspension stirred for 24 hours. SEC was then effected on a 10 mm×300 mm column packed with Toyopearl 65F eluted with 0.15M NaCl, 0.01 M NaCO₃, pH 9 at 0.75 ml/min (FIG. 6).

### b) Amination of Ficoll™ Stabilised Iron Oxide Nanoparticles Using Ammonia (IIb)

Sodium hydroxide (1.4 g) was dissolved in a solution of Ficoll-stabilised iron oxide nanoparticles (I) (30 ml), followed by the addition of epichloroydrin (3.0 ml). After 30 minutes, concentrated ammonia solution (15 ml) was added and the solution was stirred overnight.

The solution was then neutralised with 20% hydrochloric acid and concentrated by rotary evaporation to 5 ml. Size exclusion chromatography (SEC) was then effected on an XK16 column packed with Toyopearl 65F, eluted with carbonate buffer (0.2M sodium carbonate, pH10) at 1 ml/min (FIG. 7). 3 ml fractions were collected and fractions coloured dark brown were used for particle sizing by Dynamic Light Scattering (DLS) using a Malvern Nano ZS.

### c) Amination of Ficoll Stabilised Iron Oxide Nanoparticles Using 1,2-Bis(2-aminoethoxy)ethane (III)

Sodium hydroxide (1.4 g) was dissolved in a solution of Ficoll-stabilised iron oxide nanoparticles (I, 30 ml), followed by the addition of epichlorohydrin (3.0 ml). After 30 mins of stirring, 30% (v/v) solution of concentrated 1,2-Bis(2-aminoethoxy)ethane (15 ml) was added, and the solution was stirred overnight.

The solution was neutralised and subjected to SEC as in step (b)

### d) Preparation of Linker (IVa)

Aminooxy acetic acid (0.03 g, 0.33 mmol) was dissolved in acetone (2 ml). The reaction was monitored by thin layer chromatography (solvent system chloroform:methanol 4:1, acid compounds visualised by bromocresol green), Rf=0.65. IR(KCl): 2926 cm⁻¹ (b); 1728 cm⁻¹ (s); 1442 cm⁻¹ (s); 1382 cm⁻¹ (s).

To the reaction were added DCC (0.068 g, 0.33 mmol, 1 eq.), NHS (0.038 g, 0.828 mmol, 2.5 eq.) and diisopropylethylamine (200 μl, 1.5 mmol, 5 eq.). The reaction was stirred for 2 hours. The urea by-product was filtered off and the NHS-activated isopropylidene derivative (IVa) was obtained and used in the next step.

### e) Preparation of Aminooxy Functionalised Nanoparticles (V)

To a solution of amine functionalised iron oxide nanoparticles (IIa, IIb or III) (amine conc.=0.02 mmol) was added NHS-activated isopropylidene (IV) (0.2 mmol, 10eq.) and the mixture stirred at room temperature for 2 hours. Subsequently, 1 M methoxylamine (1 ml) was added and the solution stirred for 90 minutes. The solution was concentrated to 2 ml and de-salted on a sephadex G10 XK26 column (at a flow rate of 1 ml/min in HEPES buffer (0.15M NaCl, 0.01M HEPES, pH 7.2).

### f) Preparation of Oxidised Antibody e.g. MOPC IgM (VI)

Freeze-dried MOPC (5 mg in PBS) was reconstituted in 2 ml and split into four aliquots. One aliquot was then subjected to buffer exchange by centrifugal filtration (Centricon, exclusion limit=30 kDa) with acetate buffer (0.15M NaCl, 0.01M sodium acetate, pH5.5). After 5 buffer exchanges, MOPC 21 was made up to a concentration of 2.5 mg/ml in acetate buffer and cooled on ice to 0° C. To the cooled MOPC solution was added 20 mM sodium periodate (250 μl) and the resulting mixture allowed to react in the dark for 30 minutes. The reaction was quenched by addition of glycerol (2 μl, 1.5 equiv. to periodate). The reaction was subsequently dialysed against acetate buffer by centrifugal filtration (centricon, exclusion limit=30 kDa), exchanging the buffer five times.

### g) Preparation of Fluorescent Antibody Using Fluorescent Hydrazine Derivative

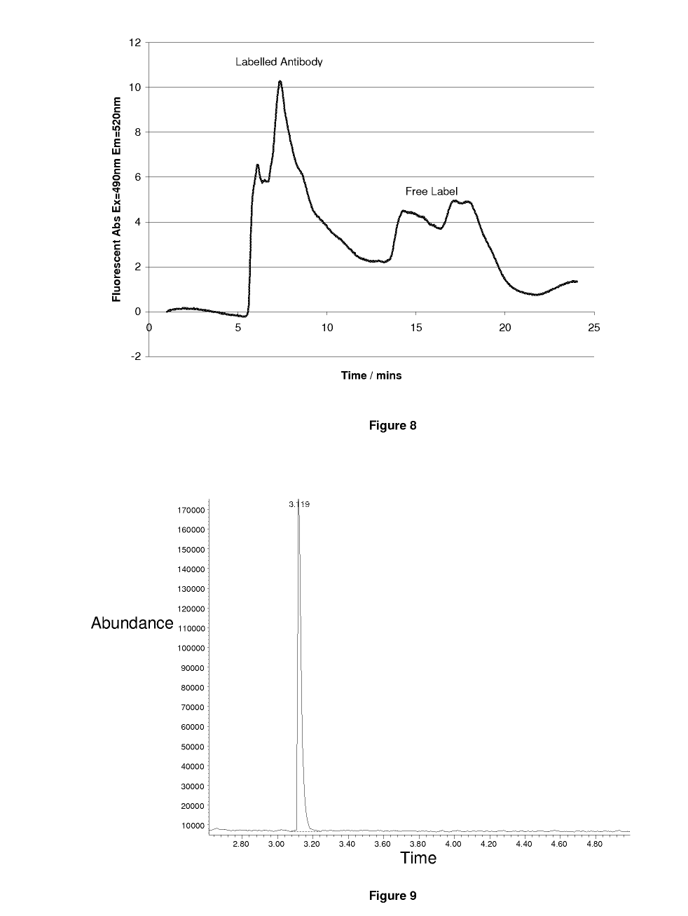

Fluorescein-5-thiosemicarbazide (40 mM in DMSO, 250 μl) was added to a freshly prepared solution of oxidized antibody (VI) (˜1.25 mg of antibody in 500 μl of acetate buffer) and allowed to react for 2 hours. To verify coupling, the product was subjected to size exclusion chromatography monitored by fluorescence (ex=490 nm, Em=520 nm) (FIG. 8). A fluorescent peak corresponding to the elution of the labelled antibody, and a second peak corresponding to the unreacted fluorescent probe were observed as shown in FIG. 8.

### h) Preparation of Nanoparticle-Antibody Conjugates (VII & VIII)

Freshly prepared antibody (VI) (200 μg in 0.15M NaCl, 0.1 M NaAc, pH 5.5, 200 μL) will be added to a solution of iron oxide nanoparticles (V) (300 μg of iron, 500 μL) and the reaction allowed to proceed overnight. Bound antibody will be separated from unbound antibody by passing the reaction mixture through a magnetic separation column (Macs, Miltenyl Biotech).

### Example 3

### a) Alternative Preparation of Linker (IVa) (see Example 2d)

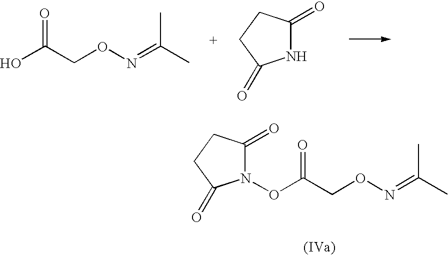

Isopropylidine aminooxy acetic acid (150 mg, 1.1 mmol) was dissolved in DMF (500 μL). To the reaction mixture was added DCC (240 mg, 1.1 mmol, 1 eq.), and NHS (180 mg, 1.6 mmol, 1.4 eq). The reaction was stirred for two hours at room temperature. The insoluble DCU product was removed by filtration and the activated ester used immediately.

### b) Alternative Preparation of Protected Aminooxy Functionalised Nanoparticles (V^{prot}) (See Example 2e)

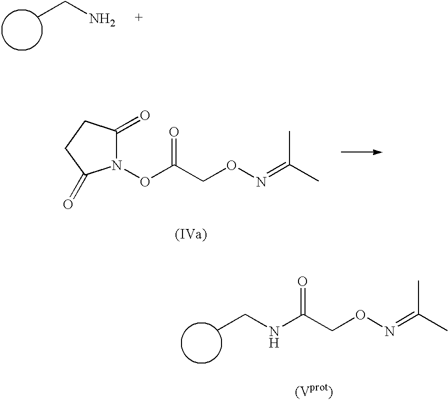

To a solution of 50 nm amine-functionalised Ficoll®-coated iron oxide nanoparticles (IIa, IIb or III) (3 mL, 0.01 mmol of primary amine groups, 0.15M sodium chloride, 0.01 M borate buffer, pH8) was added NHS-activated ester of isopropylidine aminooxy acetic acid (IVa) (˜251 mg, 1.1 mmol, 10eq.) and the reaction stirred for 1 hour. The reaction was then desalted on a Sephadex® G-10 (column: 15 mm×200 mm, 0.15M NaCl, 0.01M sodium acetate, pH5; 1 mL/min).

### c) Alternative Deprotection of Isopropylidene Protected Aminooxy Functionalised Nanoparticles (See Example 2e)

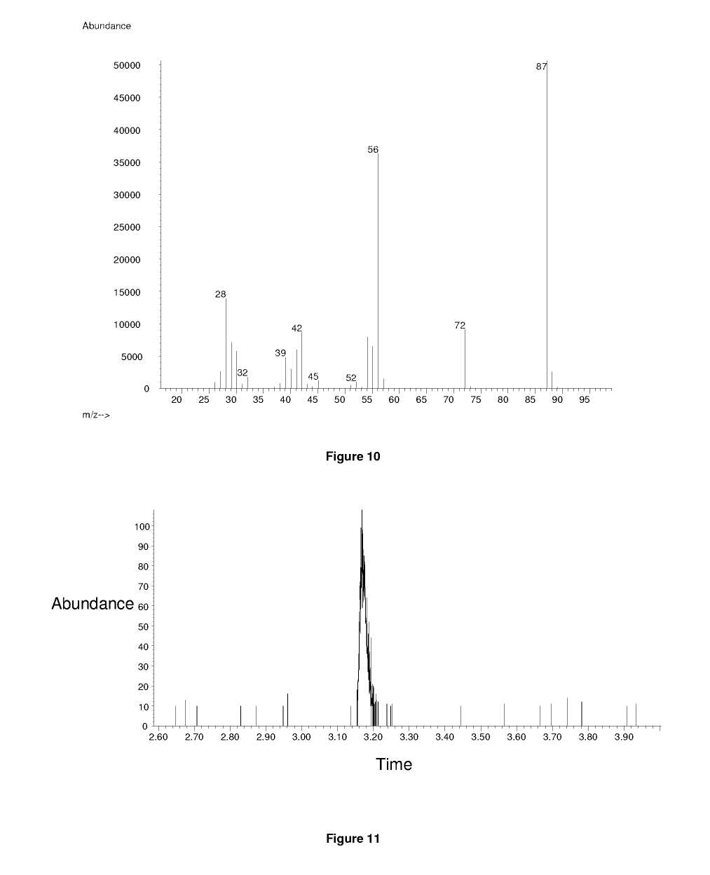

(i) To a solution of N-isopropylidene-protected aminooxy-functionalised nanoparticles (500 μL) (V^{prot}) was added 0.5M methoxyamine (500 μL, pH 4.6). The solution was kept in a gas tight sample vial for 1 hour. The headspace of the sample vial was aspirated and analysed by GC-MS. It was found that the deprotection product, propan-2-one O-methyl oxime (m/z=87), could be detected, indicating successful deprotection of isopropylidene (FIGS. 9 and 10). Given that the compound was detected immediately after the solvent peak (3.2 mins) it was postulated that propan-2-one-O-methyl oxime could be detected in situ during deprotection.

(ii) To explore the feasibility of in situ monitoring of deprotection, N-isopropylidene-protected aminooxy-functionalised nanoparticles (V^{prot}) were incubated in 0.25M methoxyamine in a sealed vial. The headspace of the deprotection reaction was then sampled and analysed by GC-MS (FIG. 11). To increase the sensitivity of monitoring, mass-selective detection (86<m/z<89) was used. A peak at an identical position to that of propan-2-one O-methyl oxime with the same principal mass was detected as shown in FIG. 10. It had therefore been demonstrated that the deprotection could be monitored in situ by GC-MS.

### d) Synthesis of Alternative Linker: 6-Isopropylideneaminooxy-Hexanoic Acid

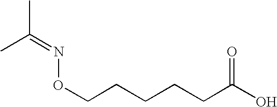

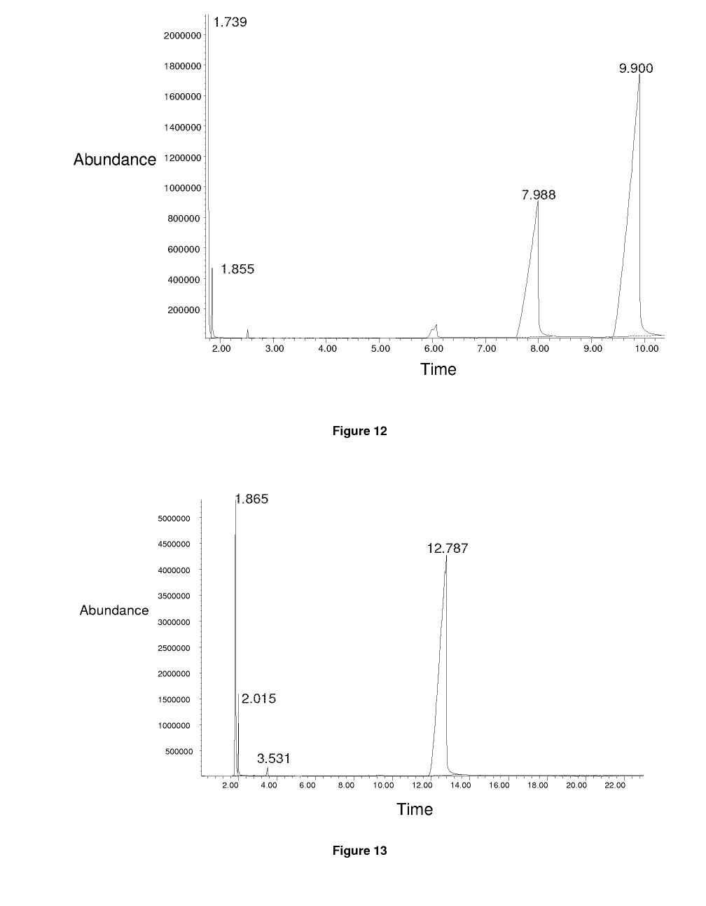

(i) 6-bromohexanoic acid (500 mg, 2.56 mmol) was dissolved in ice-cold water (2 mL). To the solution was added 40% sodium hydroxide (4 mL). The solution was stirred until all the reagents had dissolved. Finally, acetoxime (187 mg, 2.56 mmol) was added. The reaction was then passed drop wise over the course of 20 minutes through a steam-heated Leibig condenser and collected in a round bottom flask. The reaction mixture was acidified on ice to pH2 and extracted with ether. The product was analysed by GC-MS which showed that a variety of side-reactions occurred. The GC-MS spectrum is shown in FIG. 12 with the assignments shown in the table below:

| Time/mins.sec | Compound |
|---|---|
| 1.855 | 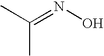 |
| 2.5 | 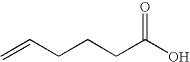 |
| 6.0 | 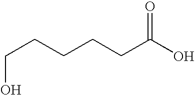 |
| 7.988 | 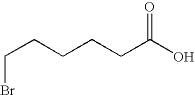 |
| 9.900 | 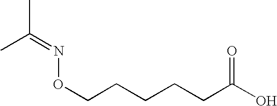 |

(ii) In an attempt to reduce the number of side reactions, as well as the reaction time, microwave heating was used. 6-bromohexanoic acid (500 mg, 2.56 mmol) was dissolved in ice water (2 mL). To the solution was added 40% sodium hydroxide (4 mL). The solution was stirred until all the reagents had dissolved. Finally, acetoxime (187 mg, 2.56 mmol) was added. Reaction mixture was then placed in a commercial microwave oven (800 W) and irradiated for 5×15 s at 75% power. The reaction mixture was acidified to pH 3 and extracted with chloroform and analysed by GC-MS. Evaporation of ether produced a colourless oil. IR(KCl): 2926 cm⁻¹ (b); 1728 cm⁻¹ (s); 1442 cm⁻¹ (s); 1382 cm⁻¹ (s). ¹H NMR (CDCl₃): 4.002 (t); 1.859 (s); 1.835 (s); 1.669 (m), 1.413 (m). Mass (EI): (Predicted/Actual) 187.12/187.

The GC-MS spectrum, shown in FIG. 13, shows that the method produced one major product. The small peak at 3.531 mins is the elimination product, and could be removed on chloroform extraction. Purity of compound was verified by ¹H NMR and ¹³C NMR; no vinylic protons were detected by ¹H NMR (3.5 ppm and 1.5 ppm)

Refluxing typically took 60 minutes, and resulted in a variety of products, whereas, microwave heating took 75 seconds and resulted in one major product.

### e) Alternative Synthesis of Fluorescently Labelled Oxidised Antibody

(i) To MOPC-21 antibody (500 μL, 1.25 mg, 0.1 M sodium carbonate, pH9) was added fluorescein isothiocyante (50 μL, 1 mg/mL in DMSO) and reacted for six hours. Unreacted fluorescein isothiocyanate was then removed by centrifugal filtration (MW cutoff −30 kDa) against 0.15M sodium chloride/0.01 M sodium acetate, pH5.5)

(ii)

To the fluorescently labelled MOPC antibody (300 μL, 1.25 mg, 0.15M sodium chloride/0.01 M sodium acetate) was added sodium periodate (230 mM, 8 μL, 0.1 M sodium acetate, pH 5.5). The reaction was carried out at 4° C. in the dark for 90 minutes. The reaction was then quenched by the addition of glycerol (10 μL). The reaction mixture was then de-salted by centrifugal filtration (MW cutoff−30 kDa) against 0.15M sodium chloride/0.01 M sodium acetate.

### f) Conjugation of Fluorescently Labelled Mopc Antibody to Nanoparticle

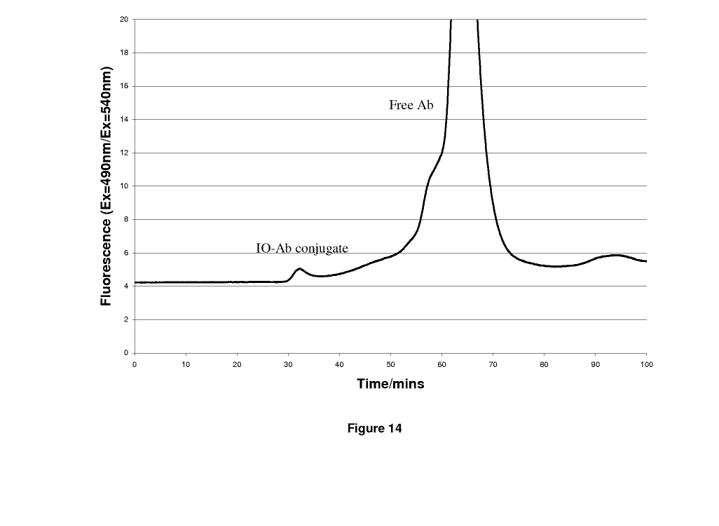

The oxidised antibody (˜1.25 mg) produced in example 3(e) was added to a solution of aminooxy-functionalised iron oxide nanoparticles (1 mL) produced by the method of example 2(e) and allowed to react overnight. The reaction mixture was then analysed by size exclusion chromatography on Superose™6 (15 mm×300 mm, flow rate of 00.1 mL/min, eluant—0.1 M sodium carbonate, pH8.5) using fluorescent detection (Ex at 490 nm/Em at 540 nm) which confirmed the conjugation of the antibody (see FIG. 14). The antibody was used in excess, hence the larger relative fluorescence of the free antibody. It is also likely that there is a decrease in fluorescence yield of the fluorescein attached to the antibody due to quenching; quenching results from proximal interactions of fluorescein molecules as the antibodies are likely to be in close proximity.

1. Iron oxide nanoparticles comprising functional groups of the formula —X—NH₂ on the outer surface of said nanoparticles, wherein X is selected from O, NR, NH or S, where R is C₁₋₇ alkyl.

2. Iron oxide nanoparticles according to claim 1, wherein X is selected from O or NH.

3. Iron oxide nanoparticles according to claim 2, wherein X is O.

4. Iron oxide nanoparticles according to claim 1, wherein the functional group is linked to the outer surface of said nanoparticle by a group: 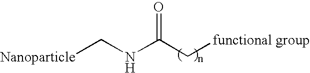
where n is 1 to 11.

5. Iron oxide nanoparticles comprising functional groups of the formula —X—NR¹R² on the outer surface of said nanoparticles, wherein X is selected from O, NH, NR or S; where R is C₁₋₇ alkyl; R¹ is H and R² is an amine protecting group, or R¹ and R² together form an amine protecting group.

6. Iron oxide nanoparticles according to claim 5, where R¹ and R² together form a dimethylimide protecting group.

7. Iron oxide nanoparticles according to claim 5, wherein X is selected from O or NH.

8. Iron oxide nanoparticles according to claim 6, wherein X is O.

9. Iron oxide nanoparticles according to claim 5, wherein the functional group is linked to the outer surface of said nanoparticle by a group: 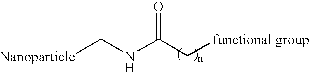
where n is 1 to 11.

10. A method of producing the nanoparticles of claim 4, comprising the step of reacting amine-functionalised iron oxide nanoparticles with a linker of formula 4, wherein Y is an activating group, n is 1 to 7 and X, R¹ and R² are as defined in claim 9.

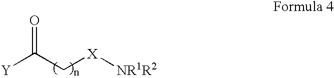

11. A method of producing the nanoparticles of claim 4, comprising the step of reacting amine-functionalised iron oxide nanoparticles with a linker of formula 4, as defined in claim 10, followed by a deprotection step to remove the amine protecting group.

12. A method according to claim 10 wherein Y is N-hydroxysuccinimide.

13. A process for attaching cell targeting ligands to the iron oxide nanoparticles of claim 1 by reaction of aldehyde groups on said ligands with said —X—NH₂ functional groups on the outer surface of the nanoparticles, to form bonds of formula —C═N—X— between the ligands and the nanoparticles.

14. A process according to claim 13, wherein the ligands are carbohydrate containing ligands

15. A process according to claim 14, wherein the carbohydrate containing ligands are glycosylated proteins.

16. A process according to claim 15, wherein the glycosylated proteins are antibodies.

17. A conjugate particle comprising an iron oxide nanoparticle linked via a bond of formula —C═N—X— to a cell targeting ligand, where X is selected from O, NR, NH or S, where R is C₁₋₇ alkyl.

18. A conjugate according to claim 17, wherein the ligand is a carbohydrate-containing ligand.

19. A conjugate according to claim 18, wherein the carbohydrate-containing ligand is a glycoprotein.

20. A conjugate according to claim 18, wherein the glycoprotein is an antibody.

21. A conjugate according to claim 17, wherein the nanoparticle is a Ficoll-stabilised iron oxide nanoparticle.

22. A conjugate according to claim 17, wherein X is selected from NH or O.

23. A conjugate according to claim 22, wherein X is O.

24. A conjugate according to claim 17, wherein the bond —C═N—X— is linked to the outer surface of said nanoparticle by a group: 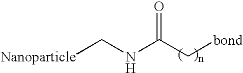
where n is 1 to 11.

25. Use of a conjugate according to claim 17 for targeting and labelling cells.

26. The use according to claim 25 for MRI contrast imaging, for cell separation, or as part of a medical treatment.
